(19) United States
(12) Patent Application Publication
(10) Pub. No.: US 2023/0081517 A1
(43) Pub. Date: Mar. 16, 2023

(54) ORALLY ADMINISTERED HYDROGEL COMPOSITION, KIT AND USE

(71) Applicant: KIFIK, Gardanne (FR)

(72) Inventors: Ouadah Hadjebi, Gardanne (FR); Hassiba Chaib-Mezrag, Sorgues (FR)

(73) Assignee: KIFIK, Gardanne (FR)

(21) Appl. No.: 17/920,038

(22) PCT Filed: Apr. 21, 2021
(86) PCT No.: PCT/EP2021/060435

§ 371 Date: Oct. 20, 2022

(30) Foreign Application Priority Data
Apr. 21, 2020 (FR) FR2003982

(51) Int. Cl. A61K 31/734; A61K 33/42; A61K 9/06; A61K 9/00; A61K 47/02

(52) U.S. Cl. CPC A61K 31/734; A61K 33/42; A61K 9/06; A61K 9/0053; A61K 47/02; A61K 9/009

(57) ABSTRACT

An orally administered hydrogel composition includes: an alginate polymer that forms a gel in aqueous solution, in the presence of a cation; an aqueous solution, in a sufficient amount; an agent for dissolving said alginate polymer in the aqueous solution; a gelation retarder; and a floating agent to form gas bubbles in the hydrogel composition the hydrogel formed by using the hydrogel composition being dissolved by using an orally administered dissolving agent. The invention applies in particular to the production of gastric implants.

### FIELD OF THE INVENTION

The present invention relates to an orally administered hydrogel composition, a kit comprising such a composition, and the use of such a composition for treating overweight or obese individuals, or for delivering active pharmaceutical or nutritional ingredients into the stomach of an individual, in a time-delayed manner.

### PRIOR ART

The prevalence of obesity, which is defined as a Body Mass Index (BMI)>30 kg/m², has doubled in 34 years. The World Health Organization (WHO) estimates that in 2016, 1.9 billion adults (over 18 years old) and 41 million children (over 5 years old) were overweight or obese. These figures have been increasing continuously ever since. It is estimated that by 2030, a record 3.3 billion adults will be overweight or obese. Obesity has a significant societal and economic impact. It is generally associated with serious or even fatal complications. It is estimated that more than 5.3 people die every minute worldwide as a direct result of obesity or being overweight.

Depending on their BMI, patients are offered three types of treatment: drug treatment, surgical treatment and gastric balloon treatment.

Treatment with a gastric balloon is generally intended for patients with moderate to severe obesity with a BMI of more than 30 and less than 40 kg/m².

Gastric balloons are inflatable medical devices, generally made of silicone, which can be filled either with a gas, a liquid or both. The procedure for inserting a balloon is simple: it is inserted in deflated form by endoscopy into the stomach of a patient. It is then filled by means of a catheter. The insertion and inflation procedure can take up to 20 minutes. After inflation the catheter is then removed. At the end of the treatment period, which lasts 3 to 6 months depending on the balloons, the balloon is removed, again by endoscopy.

However, the treatment of patients with gastric balloons according to the prior art has various disadvantages. In particular, the procedures for inserting and removing gastric balloons by endoscopy are invasive and dangerous. There have been reports of quite serious complications associated with the long-term presence of gastric balloons in the stomach. These include changes to the gastric mucosa with the formation of ulcers and/or the presence of gastric perforations, the crushing of underlying organs which sometimes result in acute pancreatitis, or intestinal obstructions in the case of balloon migration.

### SUMMARY OF THE INVENTION

In view of the above, one problem that the invention proposes to solve is to provide means for treating overweight or obese individuals which avoid the use of inflatable gastric balloons which require insertion into the stomach or removal by endoscopy.

The solution of the invention to this problem is firstly an orally administered hydrogel composition comprising:

an alginate polymer that forms a gel in aqueous solution in the presence of a cation;

a cation for polymerizing the alginate polymer in aqueous solution;

an aqueous solution, in a sufficient quantity;

a dissolving agent of said alginate polymer in the aqueous solution;

a gelation retarder; and

a floating agent for forming CO₂ bubbles in the hydrogel composition;

the hydrogel formed by means of said hydrogel composition being dissolved by means of an orally administered final dissolving agent.

In an advantageous manner:—the composition further includes an agent for strengthening the mechanical structure of the hydrogel;—the composition further includes a radio-opaque agent;—the alginate polymer is a sodium alginate polymer, and the cation is calcium;—the dissolving agent of the alginate polymer in the aqueous solution is sucrose;—the gelation retarding agent is Na₂HPO₄;—the agent for strengthening the mechanical structure of the hydrogel is selected from sorbitol, spermine, chitosan, agarose, sodium dodecyl sulfate, phosphatidylcholine, microcrystalline cellulose;—the radio-opaque agent is selected from compounds including barium, for example BaSO₄ and compounds including iodine;—the final dissolving agent is selected from citrates, calcium chelators, for example sodium citrate, citric acid or EDTA;—the floating agent is CaCO₃, glucono-δ-lactone or a microorganism; the composition comprises 0.5 to 5% sodium alginate with a viscosity of between 20-200 mPa·s, 1 to 3% CaSO₄, 0.10 to 0.20% Na₂HPO₄, 8 to 15% sucrose, 2 to 8% CaCO₃ or 0.1 to 2% yeast, 0.5% to 8% BaSO₄, and 0.5 to 8% chitosan with a viscosity between 10 and 50 mPa·s or 0.5 to 8% cellulose, the percentages being percentages by weight given in g/100 ml.

The second subject-matter of the invention is a use of a composition as defined above, for the treatment of overweight individuals who have a Body Mass Index greater than or equal to 25 kg/m².

In an advantageous manner: the use is for the treatment of obese individuals who have a Body Mass Index greater than or equal to 30 kg/m².

The third subject-matter of the invention is a use of a composition as defined above, for delivering active pharmaceutical or nutritional ingredients into the stomach of an individual in a time-delayed manner.

The fourth subject-matter of the invention comprises a composition as defined above and an orally administered dissolving agent.

### BRIEF DESCRIPTION OF THE FIGURES

The invention will be better understood from reading the following non-limiting description which is prepared with reference to the attached drawings in which:

FIG. 1 shows, in a schematic manner, the different steps for the insertion of a hydrogel according to the invention into the stomach of a patient, as well as its dissolution;

FIG. 2 is composed of 4 photographs, photographs A and C showing a hydrogel composition and a hydrogel formed from said composition, without strengthening agent, photographs B and D showing a hydrogel composition and a hydrogel formed from said composition, with strengthening agent;

FIG. 3 shows the results obtained, in terms of setting time, with hydrogel compositions according to the invention, depending on the strengthening agents it contains;

FIG. 4A shows the different steps used to determine the resistance of hydrogels to pH transitions after the ingestion of a food bolus;

FIG. 4B shows the results of the in vitro stability of hydrogels according to the invention comprising a strengthening agent or not, after the ingestion of a food bolus;

FIG. 5A shows the results obtained, in terms of weight loss, with hydrogels according to the invention, with different strengthening agents;

FIG. 5B shows the results obtained, in terms of volume loss, with hydrogels according to the invention, with different strengthening agents;

FIG. 6 shows the results obtained, in terms of weight loss, with hydrogels, in an in vitro experiment for simulating artificial digestion in the presence of natural calcium chelators;

FIG. 7A shows a first way of generating CO₂ bubbles in the hydrogel according to the invention, using the so-called calcium carbonate system;

FIG. 7B shows a second way of generating CO₂ bubbles in the hydrogel according to the invention, by using the so-called NaHCO₃+glucono-δ-lactone system;

FIG. 7C shows a third way of generating CO₂ bubbles in the hydrogel according to the invention, by using the so-called yeast/sucrose system;

FIG. 8A is a photograph which shows the floating of a hydrogel according to the invention, in an acidic solution simulating, in vitro, the acidic solution of a human stomach; and

FIG. 8B is an endoscopic view of two balloons of a hydrogel according to the invention, floating in a stomach of a mini-pig.

### DETAILED DESCRIPTION OF THE INVENTION

The invention relates to a hydrogel composition. This composition is orally administered to an individual or a human patient, in the form of a drinkable syrup. This individual is an adult for example. However, they can also be adolescents or even children of at least 5 years of age.

The composition according to the invention comprises an alginate polymer. This alginate polymer forms a gel in aqueous solution, in the presence of a cation. Advantageously, the alginate polymer is a sodium alginate polymer, the polymerization of which is initiated by calcium. The source of calcium is provided for example by CaSO₄.

The aqueous solution is water for example. It is present in the solution in a sufficient quantity for the hydrogel to form.

The composition according to the invention further includes a dissolving agent of the alginate polymer in the aqueous solution.

The composition according to the invention also includes a gelation retarder. Preferably, the gelation retarder is Na₂HPO₄.

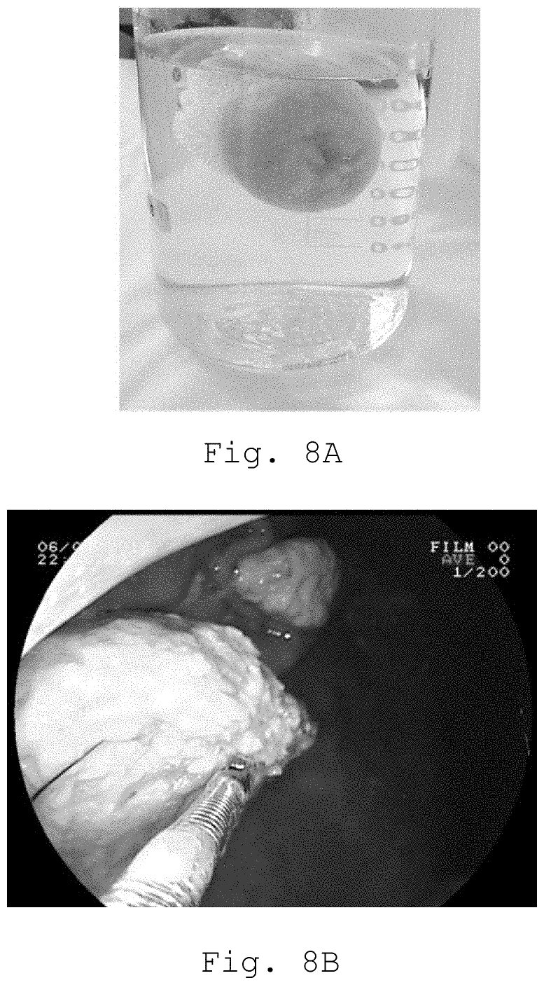

Lastly, the composition according to the invention includes a floating agent. The floating agent causes the formation of gas bubbles in the hydrogel composition. In an advantageous manner, the floating agent is CaCO₃, glucono-δ-lactone or a microorganism. The floating of the hydrogel according to the invention in an acidic solution, simulating in vitro the acidic solution of a human stomach, is illustrated in FIG. 8A. It is further illustrated in the endoscopic view shown in FIG. 8B.

The composition according to the invention comprises advantageously a strengthening agent. This strengthening agent is an agent for strengthening the mechanical structure of the hydrogel. The strengthening agent is preferably of the polymeric type, forming macromolecules which are incorporated into the hydrogel to increase its mechanical strength. Preferably, the agent for strengthening the mechanical structure of the hydrogel is selected from sorbitol, spermine, chitosan, agarose, sodium dodecyl sulfate, phosphatidylcholine, microcrystalline cellulose.

The composition according to the invention also comprises, in an advantageous manner, a radio-opaque agent. Preferably, the radio-opaque agent is selected from compounds including barium, and compounds including iodine. More preferably, the radio-opaque agent is BaSO₄.

In other words, the composition according to the invention comprises 0.5 to 5% sodium alginate with a viscosity between 20-200 mPa·s and/or 1 to 3% CaSO₄ and/or 0.10 to 0.20% Na₂HPO₄ and/or 8 to 15% sucrose and/or 2 to 8% CaCO₃ or 0.1 to 2% yeast and/or 0.5% to 8% BaSO₄ and/or 0.5 to 8% chitosan with a viscosity between 10 and 50 mPa·s or 0.5 to 8% cellulose, the percentages being weight percentages given in g/100 ml.

According to the invention, the hydrogel formed by the hydrogel composition is dissolved by means of an orally administered dissolving agent. This dissolving agent is referred to as the final dissolving agent. It is advantageously selected from citrates, calcium chelators, for example phytic acid, oxalic acid, sodium citrate, citric acid or EDTA. The dissolution of hydrogel is advantageously complete and does not produce aggregates, as decomposing products.

For the implementation of the invention, a kit is provided to a patient for example, or to a healthcare worker.

This kit comprises a first container and a second container.

The first container includes the following compounds in powder form: alginate polymer, the compound forming a cation for the polymerization of the alginate polymer, the dissolving agent of the alginate polymer, gelation retarder, floating agent, and advantageously strengthening agent, and radio-opaque agent.

The second container comprises the dissolving agent of the hydrogel, also in powder form.

The contents of the first container are dissolved in the aqueous solution to form the hydrogel composition.

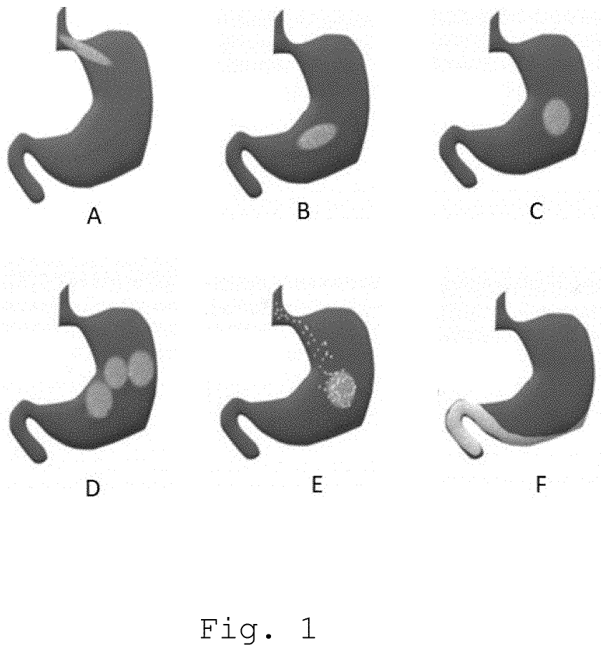

As shown in step A of FIG. 1, this hydrogel composition is drunk by the patient, after dissolving, in the manner of a syrup. In practice, the retarding agent retards the polymerization of the alginate polymer. As a result, the composition is drinkable at this stage of implementation of the invention.

The orally administered composition is then delivered to the patient's stomach via the esophagus.

The polymerization retardant has a temporary action. As shown in FIG. 1, step B, after the admission of the hydrogel composition into the stomach, for example after 2 to 3 minutes, the composition undergoes gelation in the stomach. In practice, the calcium provided by the CaSO₄ enables this gelation.

The stomach has an acidic pH, which varies over time. This acidity is due to the presence of hydrochloric acid in the stomach. The gelation is accompanied by a reaction of the floating agent. In one example, the floating agent reacts with the hydrochloric acid present in the stomach to form gas bubbles, namely CO₂, in the composition which is gelating. The gas bubbles formed are trapped in the forming gel. This gel is therefore an aerogel. It can be described as a hybrid hydrogel/aerogel. In another example, the floating agent is formed by microorganisms contained in the hydrogel composition. The microorganisms are for example yeasts, which are trapped in the gel and produce CO₂ bubbles after consuming the sucrose also contained in the gel. They perform the glycolysis of glucose to pyruvate with the release of CO₂, or the glycolysis of glucose to ethanol, in the presence of O₂. The resulting gel, which is shown in FIG. 1, step C, is essentially in the form of a substantially spherical or ovoid balloon. The gel floats in the stomach where it takes up a space which is a function of the amount of composition delivered to the stomach.

For digestion, the stomach contracts then relaxes. The strengthening agent strengthens the structure of the gel according to the invention. Its presence makes it possible to extend the time the hydrogel resides inside the stomach. It allows it to mechanically resist the contraction forces exerted by the layers of muscle in the stomach.

Alginate gels are resistant to acidic medium, and are not degraded by human α-amylase, unlike chitosan gels and starch gels. The gel according to the invention, which is established in the stomach, is stable. Its stability is maintained for several weeks or months.

As shown in FIG. 1, step D, in an advantageous manner, the hydrogel composition according to the invention is taken sequentially over a period of 3 weeks. Each week, the patient swallows a set volume of the composition which will form a balloon of 200 to 250 ml. In total, 3 balloons of 200 to 250 ml will be present in the patient's stomach. The sequential intake makes it possible for the patient to get used to the feeling of a mass in the stomach and limits some adverse events such as nausea, vomiting and abdominal pain. It is possible to check the placement and the situation of the hydrogel of the invention in the patient's stomach due to the presence of the radio-opaque agent, by simply taking an X-ray of the abdominal area of the patient's body where the stomach is located.

To remove the hydrogel, as shown in FIG. 1, step E, the dissolving agent contained in the second container of the kit according to the invention is used.

This dissolving agent is dissolved for example in an aqueous solution. It is then drunk by the patient. The solution including this agent is then fed into the stomach via the duodenum. Once in contact with the hydrogel of the invention, it dissolves it and the latter is evacuated from the patient's stomach during gastric emptying. This latter step is denoted F in FIG. 1.

According to the invention, the hydrogel composition is thus able to be used for the treatment of overweight individuals who have a Body Mass Index which is greater than or equal to 25 kg/m². In an advantageous manner, it is used for the treatment of obese individuals who have a Body Mass Index greater than or equal to 30 kg/m².

According to the invention, the hydrogel composition can be used for the delivery of active pharmaceutical or nutritional ingredients into the stomach of an individual, in a time-delayed manner. The active ingredients are advantageously contained in the hydrogel composition, then trapped in therein, and their release into the stomach is delayed.

Ultimately, the invention relates to an innovative class III intragastric device capable of reducing both the safety problems associated with the gastric balloon and also the costs. It has been developed preferably for adults with a BMI between 30 and 40 kg/m², but may be eventually offered to adolescents or children. The formulation of the gel composition according to the invention is unique, and composed of biocompatible agents. No toxic agents are used. The composition is finally administered orally, in the form of syrup. It forms a spherical or ovoid structure, aerated due to the presence of bubbles, radio-opaque when in the presence of gastric juices in particular at a pH between 2 and 3. This structure is stable for more than 4 months in the simulated intragastric environment. It retains 80% of its weight/volume at the end of the treatment. It can then be completely dissolved without forming aggregates after a few hours via the second solution, similarly aqueous, also administered orally and consisting of a food additive.

### EXAMPLE 1: PREPARATION Of A HYDROGEL

The hydrogel composition was prepared according to the following invention, the percentages are given in weight relative to volume w/v:

| | |
|---|---|
| Na-alginate | 2% |
| CaSO₄ | 1.75% |
| Na₂HPO4 | 0.16% |
| sucrose | 12% |
| CaCO₃ | 1% |
| water | 83.09%. |

For the preparation of this hydrogel, all of the ingredients were mixed in the form of powder in a beaker and then water was added to obtain a volume of hydrogel of 250 mL corresponding to a balloon internalized in one intake by a patient.

### EXAMPLE 2: ANOTHER EXAMPLE OF HYDROGEL COMPOSITION

The hydrogel composition was prepared according to the following invention, wherein the percentages are given in weight relative to volume w/v:

| | |
|---|---|
| Na-alginate | 2% |
| CaSO₄ | 1.75% |
| Na₂HPO₄ | 0.14% |
| sucrose (D+) | 12% |
| CaCO₃ | 5% |
| Chitosan | 0.5-1% |
| Cellulose | 0.5-1% |
| BaSO₄ | 5% |
| water | q.s. |

### EXAMPLE 2: STRENGTHENING AGENT OF THE HYDROGEL

It should be noted that the scientific literature is poor regarding the compressive forces encountered inside the lumen of the human stomach. According to a first document, these forces do not exceed 13 kPa. According to a second document, during digestion, these forces vary between 5 kPa and 67 kPa. According to a third document, they are on average 96±12 Pa for a fed human stomach. The mechanical properties of the hydrogel according to the invention were evaluated by static compression tests by means of a Lloyd™ LRX PLUS material compressive strength measuring machine. Before this, the parameters of this machine were optimized according to the properties of the gels and in particular the dimensions of the gels, the range of forces, the deformation range, the maximum deformation. A preload of 0.5 N and a compression speed of 10 mm/min were selected.

The hydrogel of example 1 has been tested. Before breakage, this gel which does not include strengthening agent, is able to withstand an average stress of 1342±50 Pa corresponding to an average deformation of 26±9% of its length.

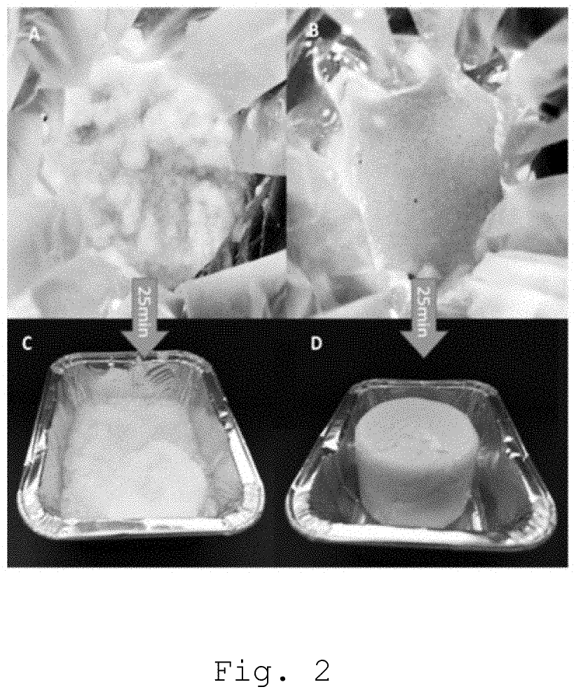

The photos in FIG. 2 show on the left photograph C a gel obtained according to the hydrogel composition of the invention according to Example 1, without a strengthening agent (chitosan), and on the right, photograph D, a gel having the same composition, but including chitosan as a strengthening agent. As can be seen in these photographs, the strengthening agent allows the final gel to be maintained in the desired form after 25 min.

The strengthening agents contained in the table below were introduced into the gel of example 1 at concentrations ranging from 0.1 to 20% w/v. A summary of the gels produced and their stress/strain data can be found in the table below.

TABLE 1

| | | Max. | Max. | |
|---|---|---|---|---|
| | | pressure | tension | |
| | Concentration | before | before | Formation |
| Strengthening agent | (% w/v) | break (Pa) | break (%) | of a gel? |
| Sorbitol | 12 | 892 | 18 | Yes - 2 |
| | | | | phases |
| Spermine | 0.1 | | | No |
| | 1 | 1409 | 24 | Yes |
| Chitosan (high | 0.1 | | | No |
| viscosity) | 1 | 4361 | 28 | Yes |
| Chitosan (low | 0.1 | | | No |
| viscosity) | 1 | 1673 | 23 | Yes |
| | 5 | 9499 | 32 | Yes |
| | 10 | 6322 | 29 | Yes |
| Agarose | 1 | 1678 | 25 | Yes |
| | 5 | 8287 | 35 | Yes |
| | 10 | 7549 | 32 | Yes |
| Cellulose | 10 | 12273 | 41 | Yes |
| Barium sulfate | 5 | 3886 | 26 | Yes |
| | 20 | 6182 | 24 | Yes |
| | 80 | 5961 | 27 | Yes |
| Calcium carbonate | 20 | 4362 | 28 | Yes |
| Sodium dodecyl | 0.1 | 1348 | 26 | Yes |
| sulfate (SDS) | 1 | 2773 | 23 | Yes |
| Phosphatidylcholine | 0.1 | 658 | 14 | Yes |
| | 10 | 833 | 21 | Yes |
| | 10 | 1314 | 22 | Yes |
| Chitosan LV - | 5-5 | 9126 | 26 | Yes |
| barium sulfate | | | | |

### EXAMPLE 3: GELATION TIMES OF HYDROGELS COMPRISING A STRENGTHENING AGENT

In this example, gelation times were determined for alginate gel compositions comprising a strengthening agent, and which are capable of resisting a maximum stress of at least 4000 Pa. This gelation time should not be less than 5 minutes so that the patient has the time to drink the composition according to the invention and it is administered into the stomach. These compositions are those of example 2, which comprise chitosan HV (0.1 and 1% w/v) and LV (0.1, 1.5 and 10% w/v), agarose (1.5 and 10% w/v), cellulose (10% w/v), barium sulfate (1.5, 10 and 20% w/v), calcium carbonate (20% w/v) and chitosan LV-barium sulfate (both at 5% w/v). First of all, the setting time of these strengthened gels was determined. For this purpose, once mixed, the powders are added to distilled water then the solution is stirred with a spatula for several seconds. The aqueous dispersion is stirred at ambient temperature on a rocker plate set at 10 rpm. Every minute the container is inclined 90° to verify whether the solution is still flowing or not. The measurements were stopped after 25 minutes. This time limit was selected by taking into account that half of the stomach emptying time after water ingestion is 13±1 min.

The results are shown in FIG. 3. As can be seen in this figure, the chitosan, in particular LV, is of greatest interest as it has little impact on the setting time and produces good mechanical strength. The calcium carbonate also has very little impact on the gelation kinetics but promotes less mechanical strength. On the contrary, the cellulose and barium sulfate induce high resistance to stress/strain but trigger rapid gelation. The barium sulfate is also of interest as a contrast agent for radioscopy and computed tomography. Lastly, a composite based on chitosan LV and barium sulfate has good mechanical properties but forms gel too quickly. However, as it combines the mechanical properties of chitosan and the contrast properties of barium sulfate, this composite could be a good compromise.

### EXAMPLE 4: STABILITY OF HYDROGELS IN VITRO AND IMPACT OF The Strengthening Agent

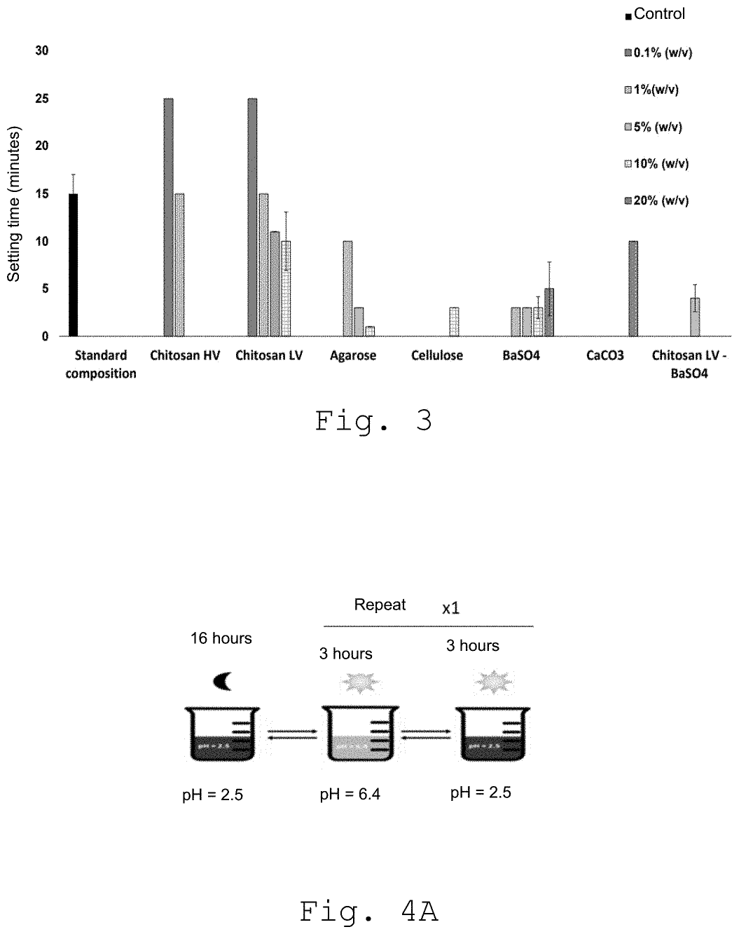

Hydrogels according to the invention were placed in a simulated gastric juice for 4 months, oscillating from an extremely acidic pH, pH=2.4 for 3 hours or 16 hours, to a quasi-neutral pH of 6.4 for 3 hours as shown in FIG. 4A. Two transitions per day (3 hours->3 hours->3 hours->3 hours->16 hours) were performed for 4 consecutive months.

In FIG. 4B, which illustrates the results obtained in terms of weight loss in percentages over time, hydrogel 1 is a hydrogel including the alginate polymer polymerized by the cation in aqueous solution in the presence of a strengthening agent. The hydrogel 2 is the hydrogel of Example 2, comprising a strengthening agent. The hydrogel 3 is formed by a polymerized alginate, without strengthening agent, including a radio-opaque agent, namely BaSO₄. The hydrogel 4 is formed by a polymerized alginate, without strengthening agent and without radio-opaque agent.

As can be seen from the curves shown in FIG. 4B, only hydrogels 1 and 2 including a strengthening agent show a more or less constant resistance over time over the 4 months of the experiment. In the absence of such an agent, the stability of the hydrogel is not ensured over time.

### EXAMPLE 5: STABILITY OF HYDROGELS ACCORDING TO PH

The physicochemical stability of hydrogels according to the invention in a simulated gastric liquid (pH 2.5 and 6.4) was evaluated by measuring their weight and their volume each week for a period of six weeks. Four gel compositions were compared in this study: the base composition of example 1, as well as compositions including chitosan LV 10% w/v, agarose 10% w/v, BaSO4 10% (w/v).

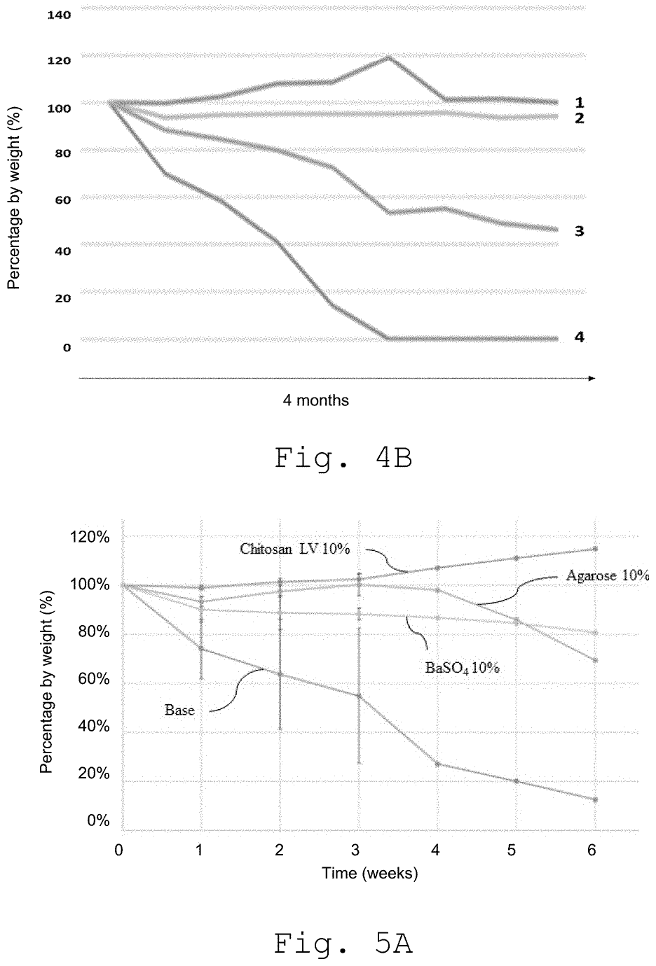

As shown in FIGS. 5A and 5B, the composition of Example 1 (10% w/v) shows a rapid but variable decrease in weight and a slower decrease in volume. This evolution over time corresponds to the floating ability of these gels. The composition comprising chitosan LV (10% w/v) is of interest, and it is found that the weight and the volume increase slowly over time. However, the hydrogel formed in this way is not capable of floating. The hydrogel including agarose (10% w/v) is somewhat stable for four weeks before a degradation in weight and volume is observed. This gel is not capable of floating, like the preceding gel. The hydrogel including barium sulfate (10% w/v) has a slow and steady decrease in weight and volume and like other gels it is not capable floating.

### EXAMPLE 6: EX-VIVO STABILITY AND IMPACT OF THE STRENGTHENING AGENT: ARTIFICIAL IN VITRO DIGESTION

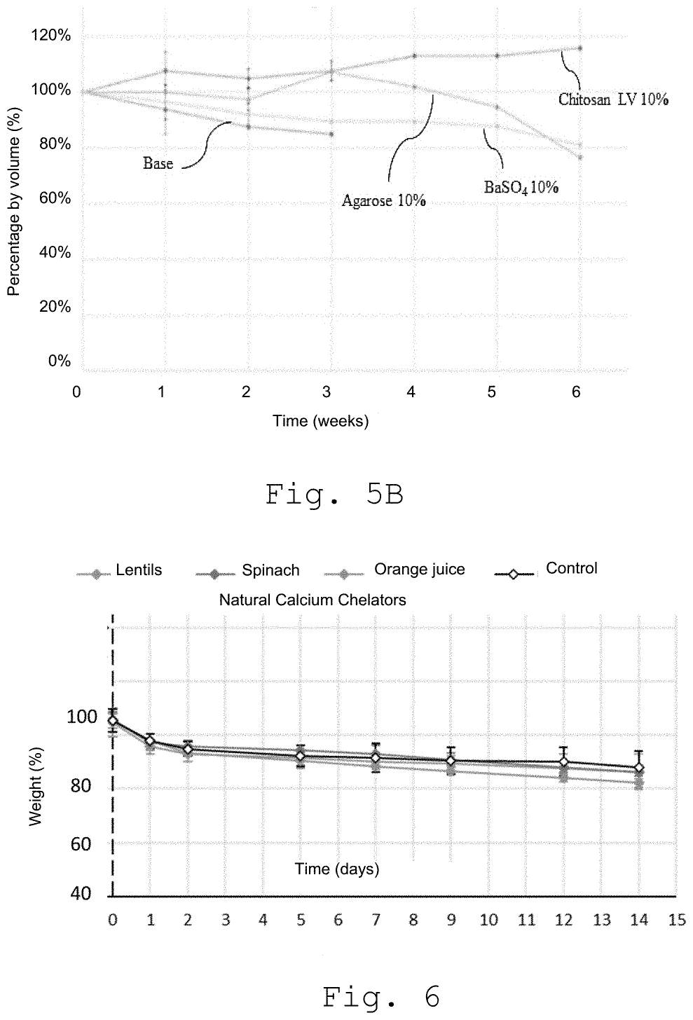

The hydrogel composition according to Example 2 was tested for stability by means of artificial in vitro digestion tests. An amount of hydrogel corresponding to a volume of 50 mL was placed at 37° C. for 14 consecutive days under agitation in a digestion buffer, at pH=3, with or without food containing high levels of calcium chelators, namely lentils, which contain phytic acid, spinach, which contain oxalic acid, or orange juice, which contains citric acid. As shown in FIG. 6, the hydrogel according to the invention has a perfect resistance to extreme conditions related to digestion (acidic pH and natural calcium chelators provided by the food), the control being performed when said hydrogel does not include calcium chelators.

### EXAMPLE 7: FLOATABILITY OF THE HYDROGEL

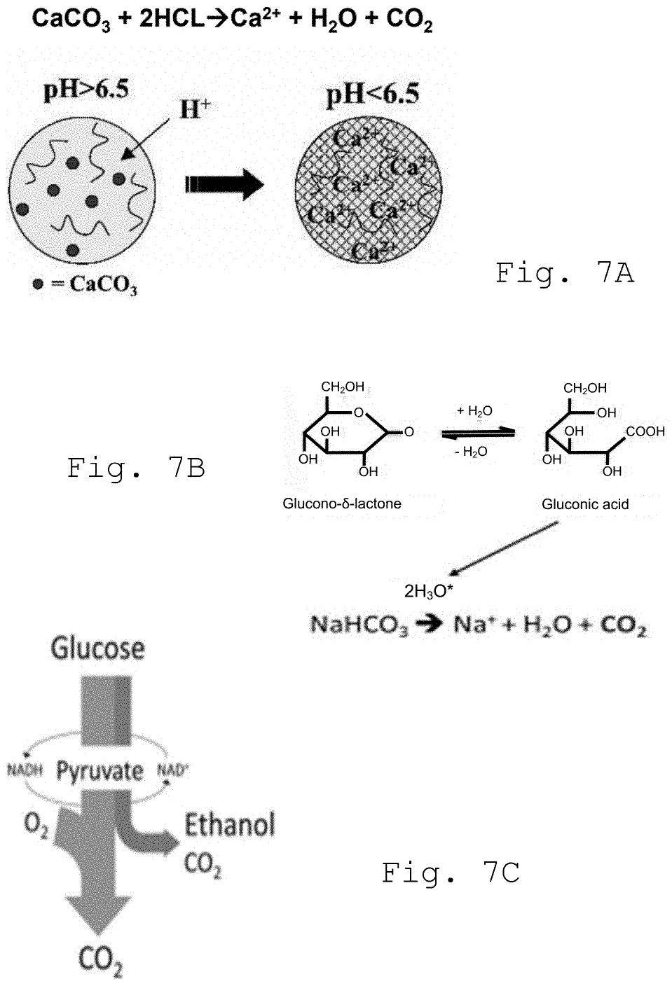

The foaming and swelling capabilities of the hydrogels according to the invention are related to the content and to the reactivity of the floatation agents. Calcium carbonate was used successfully to produce CO₂ and aerogels. By adopting the base composition of example 1, an acidic medium proved to be sufficient to trigger the dissolution of calcium carbonate and the floating of the gels. Alternatively, the calcium carbonate system can be replaced either by a gluconolactone-sodium bicarbonate system or by a yeast-sucrose system. The three systems are shown in FIGS. 7A, 7B and 7C.

FIG. 7A is a photograph which shows the gel obtained according to the composition of Example 1, in an acidic solution comprising HCl. As shown in this figure, the gel floats, in vitro, in this acidic solution. FIG. 5B is an endoscopic view of two balloons of this same gel, in vivo, in the stomach of a mini-pig, 1 week after the ingestion of the hydrogel composition. Likewise, the hydrogel or, indeed, the hydrogel-aerogel hybrid, floats in the stomach of this mini-pig.

The calcium carbonate system is easy to use. It is safe. It has been studied with hydrogels including chitosan as a strengthening agent. All of the gels tested have been found to be mechanically stable. However, at pH 2.5, after one day, none of them were able to float, although some bubbles could be observed on their surface. On the contrary, just after their incubation in an acidic medium at pH 1.2, all of the samples tested were able to float rapidly. After 7 days, no change was observed.

The gluconolactone-sodium bicarbonate system shown in FIG. 7B is based on the hydrolysis of the lactone to produce gluconic acid which reacts in turn with sodium bicarbonate to produce CO₂. This system was tested with alginate gels strengthened with chitosan. It appears that the gels strengthened with chitosan are not significantly weakened by the foaming system and some float after the first day of incubation at pH 2.5. The presence of chitosan improves the elastic properties of the gels and prevents gas expansion-induced weakening and destruction. The swelling of gels is inversely proportional to the concentration of chitosan.

The yeast-sucrose system (Saccharomyces Cerevisiae/sucrose) is a system currently used in baking to ensure the expansion of the bread dough prior to its solidification by cooking. In this bio-fermentation process, CO₂ is produced by the consumption of sucrose followed by aerobic or anaerobic glycolysis. This system was tested with alginate gels strengthened with chitosan. For all compositions the gels are mechanically stable, probably due to the cross-kinking of the chitosan. From day one, some of the tested compositions, which contain 0.6 and 0.9% (w/v) dry yeast, are able to generate sufficient gas to form an aerogel. After 3 days of incubation, hydrogels containing 0.3% (w/v) dry yeast, were also able to float.

1. A hydrogel composition for oral administration comprising:
an alginate polymer that forms a gel in aqueous solution, in the presence of a cation;
a cation for polymerization of the alginate polymer in aqueous solution;
an aqueous solution, in a sufficient quantity;
a dissolving agent of the alginate polymer in the aqueous solution;
a gelation retarder;
a floating agent for forming bubbles of CO₂ in the hydrogel composition; and
an agent for strengthening the mechanical structure of the hydrogel,
the hydrogel formed by the hydrogel composition being adapted to be dissolved by an orally administered final dissolving agent.

2. The hydrogel composition according to claim 1, wherein the strengthening agent is a polymer, forming macromolecules incorporated in the hydrogel.

3. The hydrogel composition according to claim 1, wherein the hydrogel composition further includes a radio-opaque agent.

4. The hydrogel composition according to claim 1, wherein the alginate polymer is a sodium alginate polymer and the cation is calcium.

5. The hydrogel composition according to claim 1, wherein the dissolving agent of the alginate polymer in the aqueous solution is sucrose.

6. The hydrogel composition according to claim 1, wherein the gelation retardant is Na₂HPO₄.

7. The hydrogel composition according to claim 1, wherein the agent for strengthening the mechanical structure of the hydrogel is selected from the group consisting of sorbitol, spermine, chitosan, agarose, sodium dodecyl sulfate, phosphatidylcholine, and microcrystalline cellulose.

8. The hydrogel composition according to claim 1, wherein the radio-opaque agent is selected from the group consisting of compounds including barium and compounds including iodine.

9. The hydrogel composition according to claim 1, wherein the final dissolving agent is selected from the group consisting of citrates, calcium chelators, citric acid and EDTA.

10. The hydrogel composition according to claim 1, wherein the floating agent is selected from the group consisting of CaCO₃, glucono-δ-lactone and microorganisms.

11. The hydrogel composition according to claim 1, wherein the hydrogel composition comprises:
0.5 to 5% sodium alginate with a viscosity between 20-200 mPa·s,
1 to 3% CaSO₄,
0.10 to 0.20% Na₂HPO₄,
8 to 15% sucrose,
2 to 8% CaCO₃ or 0.1 to 2% yeast,
0.5% to 8% BaSO₄, and
0.5 to 8% chitosan with a viscosity between 10 and 50 mPa·s or 0.5 to 8% cellulose,
the percentages being weight percentages given in g/100 ml.

12. A method of treating an overweight individual having a Body Mass Index higher than or equal to 25 kg/m², the method comprising:
orally administering an effective amount of the hydrogel according to claim 1 to the individual.

13. The method according to claim 12, wherein the individual is an obese individual having a Body Mass Index greater than or equal to 30 kg/m².

14. A method of delivering an active pharmaceutical or nutritional ingredient into the stomach of an individual, the method comprising:
orally administering the active ingredient and the hydrogel composition according to claim 1 to the individual.

15. A kit comprising:
the hydrogel composition according to claim 1,
a dissolving agent adapted for oral administration.

16. A kit comprising:
the hydrogel composition according to claim 11, and
a dissolving agent adapted for oral administration.

17. The method according to claim 12, further comprising orally administering a dissolving agent.

18. The method according to claim 14, further comprising orally administering a dissolving agent.

19. The hydrogel composition according to claim 1, wherein the radio-opaque agent is BaSO₄.

20. The hydrogel composition according to claim 1, wherein the final dissolving agent is sodium citrate.
